(19) United States
(12) Patent Application Publication
(10) Pub. No.: US 2019/0060007 A1
(43) Pub. Date: Feb. 28, 2019

(54) User Control Pendant Assembly For Remotely Controlling Surgical Device

(71) Applicant: MAKO Surgical Corp., Ft. Lauderdale, FL (US)

(72) Inventor: John Fossez, Frisco, TX (US)

(73) Assignee: MAKO Surgical Corp., Ft. Lauderdale, FL (US)

(21) Appl. No.: 16/110,250

(22) Filed: Aug. 23, 2018

Related U.S. Application Data
(60) Provisional application No. 62/551,392, filed on Aug. 29, 2017.

(51) Int. Cl. A61B 34/30; A61B 18/14; A61B 34/20; G16H 20/40; G05B 19/4155

(52) U.S. Cl. CPC A61B 34/30; A61B 18/14; A61B 2018/00595; G16H 20/40; G05B 19/4155; A61B 34/20

(57) ABSTRACT

A user control pendant assembly for remotely controlling a surgical device includes a housing, a plurality of user controls associated with the housing, one of the user controls being actuated by a user during operation of the surgical device, and an accessory device coupled to the housing to allow a user to perform an ancillary function during operation of the surgical device.

### RELATED APPLICATION

This application claims priority to and the benefit of U.S. Provisional Patent Application No. 62/551,392, filed on Aug. 29, 2017, the entire disclosure of which is hereby incorporated by reference herein.

### TECHNICAL FIELD

The present invention relates generally to robotic systems and, more particularly, to a user control pendant assembly for remotely controlling a surgical device of a robotic system.

### BACKGROUND

Robotic systems used in surgery are well known. One such system is a machining system having one or more cutting tools for forming a workpiece (e.g., bone) into a desired shape by cutting material from the workpiece.

In some applications, a user control pendant may be used to adjust a feed rate of the robotic system. Generally, the user control pendant includes a processor, switches in communication with the processor, and one or more buttons associated with the switches. The processor transmits signals resulting from pressing of the buttons on the user control pendant to a manipulator controller of the robotic system and/or to convert the pressing of the buttons into coefficients representative of the user adjustment of the feed rate.

The user control pendant may also be used to start, stop, or continue certain operations of the robotic system. For instance, a button on the pendant may be required to be constantly engaged by the user in order to continue cutting operations when the cutting tool is being moved autonomously by the robotic system. In this case, if the user ceases actuating the button, then autonomous cutting ceases. Only by virtue of continuous actuation of the button can cutting operations continue. As a result, users are sometimes required to hold the user control pendant during an entire operation in which autonomous cutting is to be performed so that cutting can continue without interruption. However, the user control pendant does not allow the user to perform any other function besides controlling the robotic system.

Therefore, there is a need in the art for addressing one or more of these deficiencies.

### SUMMARY OF THE INVENTION

The present invention provides a user control pendant assembly for remotely controlling a surgical device. The user control pendant assembly comprises a housing, a plurality of user controls (such as buttons) associated with the housing, one of the user controls being actuated by a user during operation of the surgical device, and an accessory device coupled to the housing to allow a user to perform an ancillary function during operation of the surgical device, the ancillary function being ancillary to operation of the surgical device.

### BRIEF DESCRIPTION OF THE DRAWINGS

Advantages of the present invention will be readily appreciated as the same becomes better understood by reference to the following detailed description when considered in connection with the accompanying drawings.

FIG. 1 is a perspective view of a robotic system illustrated schematically with a user control pendant assembly, according to one embodiment of the present invention.

FIG. 2 is a perspective view of a waste collection system illustrated schematically with a user control pendant assembly, according to one embodiment of the present invention.

FIG. 3 is a perspective view of the user control pendant assembly, according to one embodiment of the present invention.

FIG. 4 is a side elevational view of the user control pendant assembly of FIG. 3.

FIG. 5 is a top elevational view of the user control pendant assembly of FIG. 3.

FIG. 6 is bottom elevational view of the user control pendant assembly of FIG. 3.

FIG. 7 is a front elevational view of the user control pendant assembly of FIG. 3.

FIG. 8 is a partial exploded view of the user control pendant assembly of FIG. 3.

FIGS. 9 and 9A are perspective views of the user control pendant assembly of FIG. 3 with another embodiment of an accessory device.

FIG. 10 is a perspective view of the user control pendant assembly of FIG. 3 with an accessory device removed.

FIG. 11 is a schematic view of an irrigation system illustrated schematically with a user control pendant assembly, according to one embodiment of the present invention.

### DETAILED DESCRIPTION

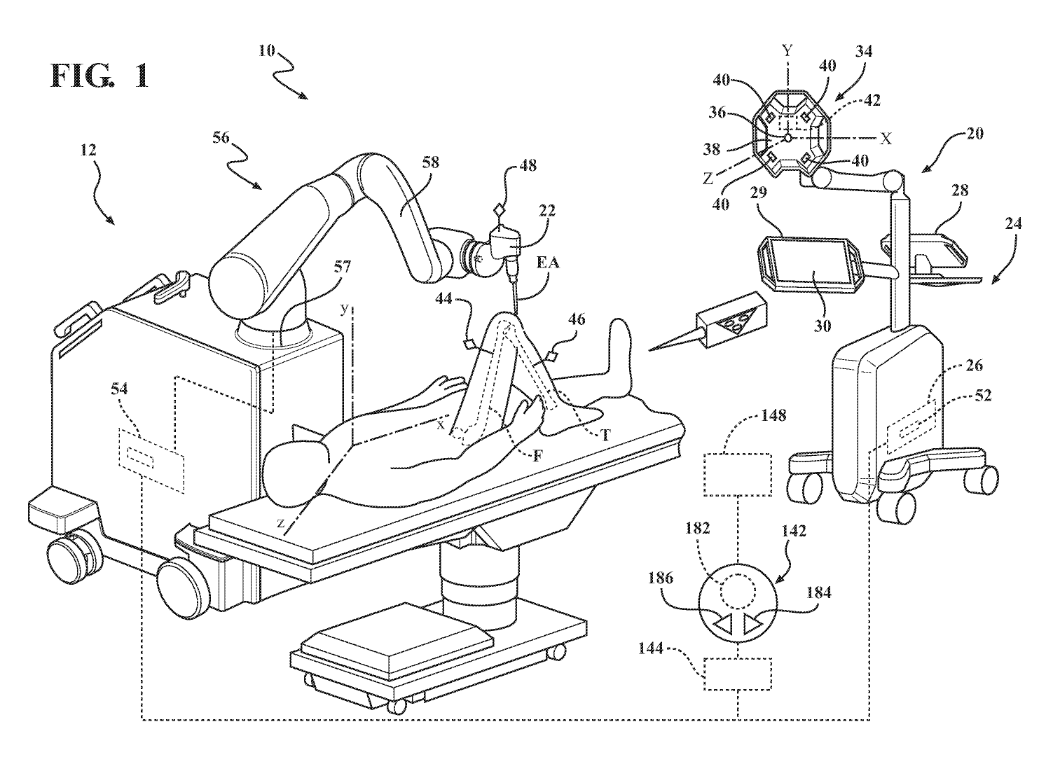

Referring to FIG. 1, a robotic system is illustrated for performing surgery on a patient. The version shown in FIG. 1 comprises a material removal system 10 for removing material from a workpiece (e.g., bone), but it should be appreciated that other types of robotic systems are also contemplated. The material removal system 10 is shown in a surgical setting such as an operating room of a medical facility. In the embodiment shown, the material removal system 10 includes a machining station 12 and a guidance station 20. The guidance station 20 is set up to track movement of various objects in the operating room. Such objects include, for example, a surgical tool 22, a femur F of a patient, and a tibia T of the patient. The guidance station 20 tracks these objects for purposes of displaying their relative positions and orientations to the surgeon and, in some cases, for purposes of controlling or constraining movement of the surgical tool 22 relative to virtual cutting boundaries associated with the femur F and tibia T.

The guidance station 20 includes a computer cart assembly 24 that houses a navigation computer 26. A navigation interface is in operative communication with the navigation computer 26. The navigation interface includes a first display 28 adapted to be situated outside of the sterile field and a second display 29 adapted to be situated inside the sterile field. The displays 28, 29 are adjustably mounted to the computer cart assembly 24. First and second input devices such as a keyboard and mouse can be used to input information into the navigation computer 26 or otherwise select/control certain aspects of the navigation computer 26. Other input devices are contemplated including a touch screen 30 or voice-activation.

A localizer 34 communicates with the navigation computer 26. In the embodiment shown, the localizer 34 is an optical localizer and includes a camera unit 36. Other types of localizers are also contemplated, including localizers that employ ultrasound, radio frequency (RF) signals, electromagnetic fields, and the like. The camera unit 36 has an outer casing 38 that houses one or more optical position sensors 40. In some embodiments at least two optical sensors 40 are employed, preferably three or four. The optical sensors 40 may be four separate charge-coupled devices (CCD). In one embodiment four, one-dimensional CCDs are employed. It should be appreciated that in other embodiments, separate camera units, each with a separate CCD, or two or more CCDs, could also be arranged around the operating room. The CCDs detect infrared (IR) signals.

The camera unit 36 is mounted on an adjustable arm to position the optical sensors 40 with a field of view of the below discussed trackers that, ideally, is free from obstructions. In some embodiments the camera unit 36 is adjustable in at least one degree of freedom by rotating about a rotational joint. In other embodiments, the camera unit 36 is adjustable about two or more degrees of freedom.

The camera unit 36 includes a camera controller 42 in communication with the optical sensors 40 to receive signals from the optical sensors 40. The camera controller 42 communicates with the navigation computer 26 through either a wired or wireless connection (not shown). One such connection may be an IEEE 1394 interface, which is a serial bus interface standard for high-speed communications and isochronous real-time data transfer. The connection could also use a company specific protocol. In other embodiments, the optical sensors 40 communicate directly with the navigation computer 26.

Position and orientation signals and/or data are transmitted to the navigation computer 26 for purposes of tracking objects. The computer cart assembly 24, display 28, and camera unit 36 may be like those described in U.S. Pat. No. 7,725,162 to Malackowski, et al. issued on May 25, 2010, entitled “Surgery System,” hereby incorporated by reference.

The navigation computer 26 can be a personal computer or laptop computer. The navigation computer 26 has the display 28, central processing unit (CPU) and/or other processors, memory (not shown), and storage (not shown). The navigation computer 26 is loaded with software. The software converts the signals received from the camera unit 36 into data representative of the position and orientation of the objects being tracked.

The guidance station 20 is operable with a plurality of tracking devices 44, 46, 48, also referred to herein as trackers. In the illustrated embodiment, one tracker 44 is firmly affixed to the femur F of the patient and another tracker 46 is firmly affixed to the tibia T of the patient. The trackers 44, 46 are firmly affixed to sections of bone. The trackers 44, 46 may be attached to the femur F and tibia T in the manner shown in U.S. Pat. No. 7,725,162, issued May 25, 2010, the entire disclosure of which is hereby incorporated by reference. The trackers 44, 46 could also be mounted like those shown in U.S. Patent Application Publication No. 2014/0200621, published on Jul. 17, 2014, entitled, “Navigation Systems and Methods for Indicating and Reducing Line-of-Sight Errors,” the entire disclosure of which is hereby incorporated by reference. In additional embodiments, a tracker (not shown) is attached to the patella to track a position and orientation of the patella. In yet further embodiments, the trackers 44, 46 could be mounted to other tissue types or parts of the anatomy.

A tool tracker 48 is firmly attached to the surgical tool 22. The tool tracker 48 may be integrated into the surgical tool 22 during manufacture or may be separately mounted to the surgical tool 22 in preparation for surgical procedures. The working end of the surgical tool 22, which is being tracked by virtue of the tool tracker 48, may be an energy applicator EA such as a rotating bur, saw blade, electrical ablation device, or the like. The energy applicator EA may be a separate component such as a bur, saw blade, ablator, or the like that is releasably connected to a handpiece of the surgical tool 22 or may be integrally formed with the handpiece.

The trackers 44, 46, 48 can be battery powered with an internal battery or may have leads to receive power through the navigation computer 26, which, like the camera unit 36, preferably receives external power.

In the embodiment shown, the surgical tool 22 is attached to a manipulator 56 of the machining station 12. Such an arrangement is shown in U.S. Pat. No. 9,119,655, issued Sep. 1, 2015, entitled, “Surgical Manipulator Capable of Controlling a Surgical Instrument in Multiple Modes,” the entire disclosure of which is hereby incorporated by reference.

The optical sensors 40 of the localizer 34 receive light signals from the trackers 44, 46, 48. In the illustrated embodiment, the trackers 44, 46, 48 are active trackers. In this embodiment, each tracker 44, 46, 48 has at least three active tracking elements or markers for transmitting light signals to the optical sensors 40. The active markers can be, for example, light emitting diodes or LEDs 50 transmitting light, such as infrared light. The optical sensors 40 preferably have sampling rates of 100 Hz or more, more preferably 300 Hz or more, and most preferably 500 Hz or more. In some embodiments, the optical sensors 40 have sampling rates of 8000 Hz. The sampling rate is the rate at which the optical sensors 40 receive light signals from sequentially fired LEDs (not shown). In some embodiments, the light signals from the LEDs are fired at different rates for each tracker 44, 46, 48.

Each of the LEDs are connected to a tracker controller (not shown) located in a housing of the associated tracker 44, 46, 48 that transmits/receives data to/from the navigation computer 26. In one embodiment, the tracker controllers transmit data on the order of several Megabytes/second through wired connections with the navigation computer 26. In other embodiments, a wireless connection may be used. In these embodiments, the navigation computer 26 has a transceiver (not shown) to receive the data from the tracker controller.

In other embodiments, the trackers 44, 46, 48 may have passive markers (not shown), such as reflectors that reflect light emitted from the camera unit 36. The reflected light is then received by the optical sensors 40. Active and passive arrangements are well known in the art.

In some embodiments, the trackers 44, 46, 48 also include a gyroscope sensor and accelerometer, such as the trackers shown in U.S. Pat. No. 9,008,757, issued on Apr. 14, 2015, entitled, “Navigation System Including Optical and Non-Optical Sensors,” the entire disclosure of which is hereby incorporated by reference.

The navigation computer 26 includes a navigation processor 52. It should be understood that the navigation processor 52 could include one or more processors to control operation of the navigation computer 26. The processors can be any type of microprocessor or multi-processor system. The term processor is not intended to limit the scope of any embodiment to a single processor.

The camera unit 36 receives optical signals from the LEDs of the trackers 44, 46, 48 and outputs to the processor 52 signals relating to the position of the LEDs of the trackers 44, 46, 48 relative to the localizer 34. Based on the received optical (and non-optical signals in some embodiments), navigation processor 52 generates data indicating the relative positions and orientations of the trackers 44, 46, 48 relative to the localizer 34.

Prior to the start of the surgical procedure, additional data are loaded into the navigation processor 52. Based on the position and orientation of the trackers 44, 46, 48 and the previously loaded data, the navigation processor 52 determines the position of the working end of the surgical tool 22 (e.g., the centroid of a surgical bur) and the orientation of the surgical tool 22 relative to the tissue against which the working end is to be applied. In some embodiments, the navigation processor 52 forwards these data to a manipulator controller 54. The manipulator controller 54 can then use the data to control the manipulator 56 as described in U.S. Pat. No. 9,119,655, issued Sep. 1, 2015, entitled, “Surgical Manipulator Capable of Controlling a Surgical Instrument in Multiple Modes,” the entire disclosure of which is hereby incorporated by reference.

In one embodiment, the manipulator 56 is controlled to stay within a preoperatively defined boundary set by the surgeon, which defines the material of the femur F and tibia T to be removed by the surgical tool 22. The boundary may be defined within a virtual model of the femur F and tibia T and be represented as a mesh surface, constructive solid geometry (CSG), voxels, or other boundary representation techniques.

The navigation processor 52 also generates image signals that indicate the relative position of the working end to the tissue. These image signals are applied to the displays 28, 29. The displays 28, 29, based on these signals, generate images that allow the surgeon and staff to view the relative position of the working end to the surgical site. The displays, 28, 29, as discussed above, may include a touch screen or other input/output device that allows entry of commands.

In the embodiment shown in FIG. 1, the surgical tool 22 forms part of an end effector of the manipulator 56. The manipulator 56 has a base 57, a plurality of links 58 extending from the base 57, and a plurality of active joints (not numbered) for moving the surgical tool 22 with respect to the base 57. The links 58 may form a serial arm structure as shown in FIG. 1, a parallel arm structure (not shown), or other suitable structure. The manipulator 56 has the ability to operate in a manual mode in which a user grasps the end effector of the manipulator 56 in order to cause movement of the surgical tool 22 (e.g., directly, through force/torque sensor measurements that cause active driving of the manipulator 56, or otherwise) or a semi-autonomous mode in which the surgical tool 22 is moved by the manipulator 56 along a predefined tool path (e.g., the active joints of the manipulator 56 are operated to move the surgical tool 22 without requiring force/torque on the end effector from the user). An example of operation in a semi-autonomous mode is described in U.S. Pat. No. 9,119,655, issued Sep. 1, 2015, entitled, “Surgical Manipulator Capable of Controlling a Surgical Instrument in Multiple Modes,” the entire disclosure of which is hereby incorporated by reference. A separate tracker (not shown) may be attached to the base 57 of the manipulator 56 to track movement of the base 57.

In one embodiment, when the manipulator 56 is operated in the semi-autonomous mode, the manipulator 56 is capable of moving the surgical tool 22 free of operator assistance. Free of operator assistance may mean that an operator/user does not physically contact the surgical tool 22 to move the surgical tool 22. Instead, the operator may use some form of remote control to control starting and stopping of movement. For example, the operator may hold down a button of the remote control to start movement of the surgical tool 22 and release the button to stop movement of the surgical tool 22.

In the manual mode, in one embodiment, the operator physically contacts the end effector to cause movement of the surgical tool 22, as described in U.S. Pat. No. 9,119,655, issued Sep. 1, 2015, entitled, “Surgical Manipulator Capable of Controlling a Surgical Instrument in Multiple Modes,” the entire disclosure of which is hereby incorporated by reference.

The manipulator controller 54 can use the position and orientation data of the surgical tool 22 and the patient's anatomy to control the manipulator 56 as described in U.S. Pat. No. 9,119,655, issued Sep. 1, 2015, entitled, “Surgical Manipulator Capable of Controlling a Surgical Instrument in Multiple Modes,” the entire disclosure of which is hereby incorporated by reference.

The manipulator controller 54 may have a central processing unit (CPU) and/or other manipulator processors, memory (not shown), and storage (not shown). The manipulator controller 54, also referred to as a manipulator computer, is loaded with software as described below. The manipulator processors could include one or more processors to control operation of the manipulator 56. The processors can be any type of microprocessor, multi-processor, and/or multi-core processing system. The term processor is not intended to limit any embodiment to a single processor.

The manipulator 56 may be in the form of a conventional robotic system or other conventional machining apparatus, and thus the components thereof shall not be described in detail.

The manipulator controller 54 determines the desired location to which the surgical tool 22 should be moved. Based on this determination, and information relating to the current location (e.g., pose) of the surgical tool 22, the manipulator controller 54 determines the extent to which each of the plurality of links 58 needs to be moved in order to reposition the surgical tool 22 from the current location to the desired location. The data regarding where the plurality of links 58 are to be positioned is forwarded to joint motor controllers (not shown) (e.g., one for controlling each motor) that control the active joints of the manipulator 56 to move the plurality of links 58 and thereby move the surgical tool 22 from the current location to the desired location.

As illustrated in FIG. 1, a user control pendant assembly 142, according to one embodiment of the present invention, is used to interface with the manipulator controller 54. The user control pendant assembly 142 includes a processor or pendant controller 144. The pendant controller 144 may have a central processing unit (CPU) and/or other pendant processors, memory (not shown), and storage (not shown). The pendant controller 144 is in communication with the manipulator controller 54. The pendant controller 144 is also in communication with switches (not shown) associated with user controls such as buttons 182, 184, and 186. The pendant processor could include one or more processors to transmit signals resulting from pressing of buttons 182, 184, and 186 on the user control pendant assembly 142 to the manipulator controller 54 and/or to convert the pressing of the buttons 184 and 186 into coefficients representative of the user adjustment of the feed rate. The processors can be any type of microprocessor or multi-processor system. It should be appreciated that the term processor is not intended to limit any embodiment to a single processor.

Once the practitioner is ready to begin autonomous advancement of the surgical tool 22, in the semi-autonomous mode, for example, the practitioner depresses button 182. In some versions, based on the depression of buttons 184 and 186, a coefficient representative of the user adjustment of the feed rate is transmitted from the pendant controller 144 associated with the user control pendant assembly 142 to the manipulator controller 54.

In some versions, the coefficient is 0.0, 0.25, 0.40, 0.70 or 1.0. This is the coefficient that is applied to a feed rate calculator (not shown) as the user adjustment input. In one embodiment, each depression of the button 184 results in the pendant controller 144 readjusting the feed rate coefficient down a level. Each depression of the button 186 results in the pendant controller 144 readjusting the feed rate coefficient up to the next higher level. It should be appreciated that the pendant controller 144 continually monitors the user control pendant assembly 142 to determine whether or not the buttons 182, 184, or 186 are depressed.

In one embodiment, the initial depression or depressions of the button 186 causes the pendant controller 144 to upwardly adjust the level of the user adjustment coefficient applied to the feed rate calculator. The level to which this coefficient is set is a function of the number of times the button 186 is pulsed. The feed rate calculator, in response to receipt of this non-zero coefficient, outputs a non-zero tool feed rate. This assumes that none of the other coefficients applied to feed rate calculator are zero. Based on this indication that the surgical tool 22 is to advance at a non-zero speed, the manipulator controller 54 cooperates to output signals to the joint motor controllers to cause the advancement of the surgical tool 22 along the tool path. An example of a user control pendant is disclosed in U.S. Patent Application Publication No. 2017/0000572, published Jan. 5, 2017, to Moctezuma de la Barrera et al. and entitled “Robotic Systems and Methods for Controlling a Tool Removing Material from a Workpiece”, the entire disclosure of which is hereby incorporated by reference.

The user control pendant assembly 142 may incorporate other functionality, for example, applying suction and/or irrigation, and/or cautery to stop bleeding, and/or other type of integrated tool. In one embodiment, as illustrated in FIG. 1, the user control pendant assembly 142 is connected to a source of suction 148 to allow the user to apply suction during a surgical procedure while holding the user control pendant assembly 142. In this case, the application of suction during the surgical procedure is an ancillary function of the user control pendant assembly 142, as it is ancillary to the operation of the manipulator 56 and, by extension, ancillary to operation of the surgical tool 22 connected to the manipulator 56. It should be appreciated that the user control pendant assembly 142 may be used in other systems besides a robotic system or the material removal system 10.

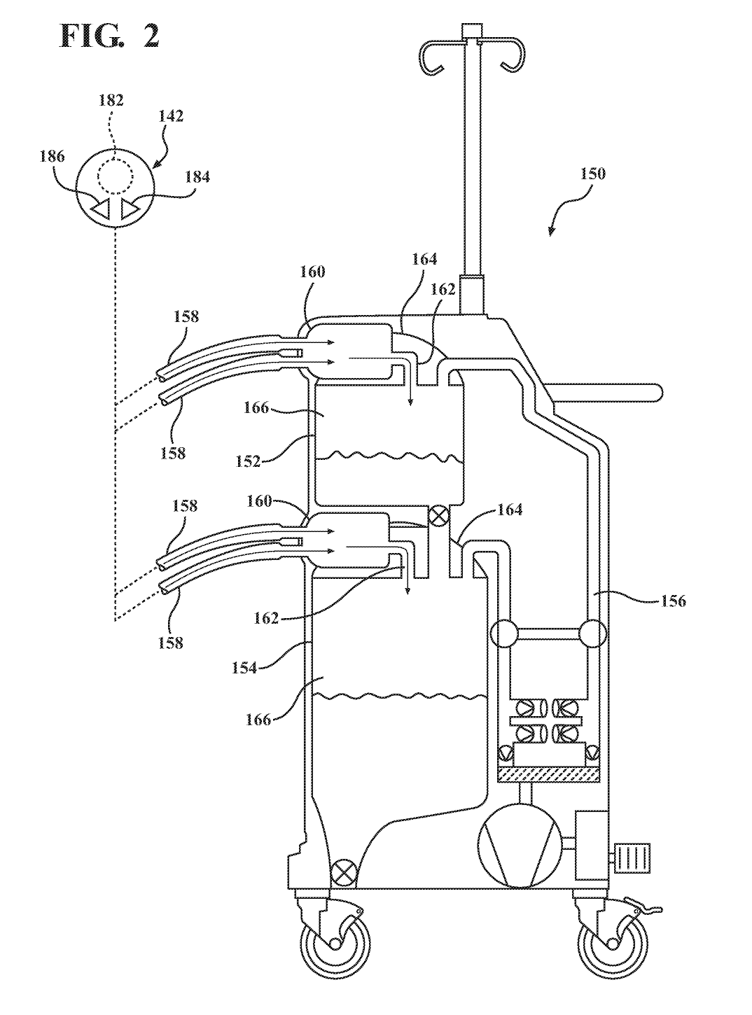

Referring to FIG. 2, the user control pendant assembly 142 may be used with a suction source from a waste collection system. For example, in this embodiment, the user control pendant assembly 142 is connected to a mobile waste collection unit 150 to apply suction during a surgical procedure while holding the user control pendant assembly 142 to control the manipulator 56 of FIG. 1. The waste collection unit 150 collects the waste material generated during the surgical procedures. For convenience, the waste collection unit 150 may also be referred to as a rover 150. An example of such a waste collection unit 150 is disclosed in U.S. Pat. No. 8,740,866 to Reasoner et al., issued Jun. 3, 2014, entitled “Medical/Surgical Waste Collection and Disposal System Including a Rover and a Docker, the Docker having Features Facilitating the Alignment of the Docker with the Rover”, the entire disclosure of which is hereby incorporated by reference. The waste collection unit 150 includes waste containers 152, 154 and a vacuum circuit 156 connected to the waste containers 152, 154. The user control pendant assembly 142 is connected by suction lines 158 to the waste containers 152, 154. In the waste collection unit 150, a vacuum is pulled in each of the waste containers 152, 154 with the vacuum circuit 156 to draw the waste material into the waste containers 152, 154 from the sites in proximity to the patient. With the vacuum present, waste material is drawn through the suction lines 158, disposable manifolds 160, and finally through waste ports 162 defined by caps 164 to enter canisters 166 of the waste containers 152, 152. It should be appreciated that users can select to simultaneously collect waste material in both waste containers 152, 154 or one at a time. It should also be appreciated that the buttons 184 and 186 on the user control pendant assembly 142 may control the vacuum circuit 156.

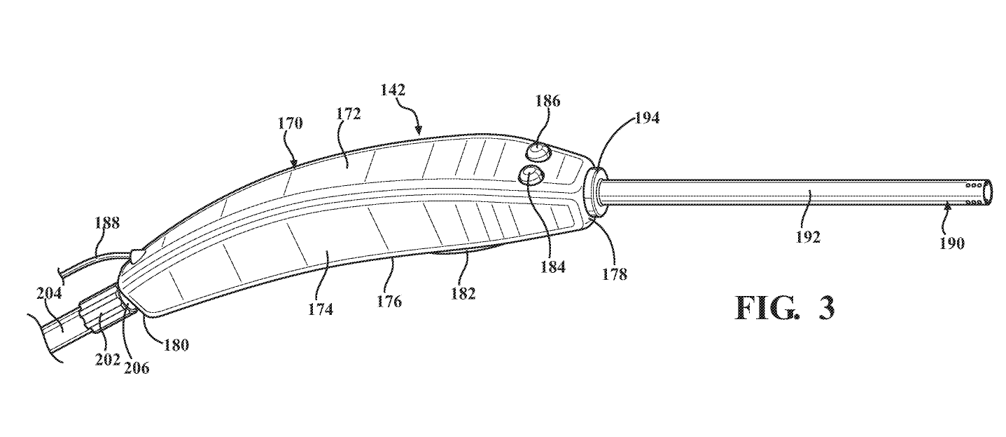

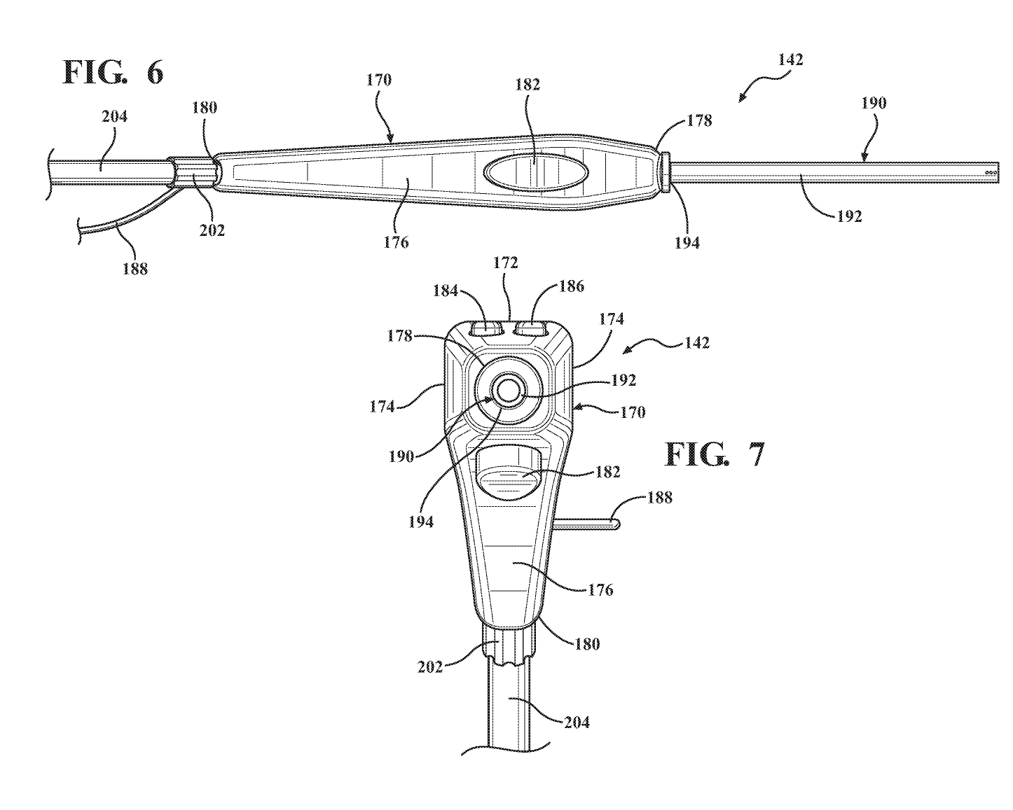

Referring to FIGS. 3-8, one embodiment of the user control pendant assembly 142 is shown in more detail. In this embodiment, the user control pendant assembly 142 includes a housing, generally indicated at 170, extending along an axis A. The housing 170 may be gently curved and ergonomic in shape to ease grasping of the housing 170 by the user, but may be any suitable shape. The housing 170 has a top 172, sides 174, and a bottom 176. The housing 170 also has a front or distal end 178 and a rear or proximal end 180. In one embodiment, the housing 170 is made of a plastic material, but can be made of any suitable material. It should be appreciated that the housing 170 is used to enclose or house the pendant controller, including the central processing unit (CPU) and/or other pendant processors (not shown), switches (not shown), memory (not shown), and storage (not shown).

The user control pendant assembly 142 may include one or more user controls (e.g., buttons) associated with the switches. In one embodiment, the user control pendant assembly 142 includes a button 182 located on the bottom 176 of the housing 170 associated with and in communication with one of the switches, which is, in turn, in communication with the pendant controller. The button 182 may also be referred to as a trigger that is held and actuated by the user at all times the manipulator 56 is machining. In other words, in order to continue operation of the manipulator 56, the user is required to constantly depress or otherwise actuate the button 182. For example, during autonomous machining of material (e.g. bone or other tissue) from the patient, to enable continuous movement of the energy applicator EA along the tool path, the user is required to continually depress the button 182. If the button 182 is not depressed, then the manipulator 56 ceases operation and cutting of material. Only when the button 182 is again depressed does autonomous cutting operations continue. In other versions, the button 182 may operate as a start/stop button so that continued depression is not required, and starting/stopping operation can be caused by a single pulse of the button 182. In this case, the user may still desire to hold the user control pendant assembly 142 while the energy applicator EA is moving autonomously along the tool path to provide immediate access to the user control pendant assembly 142 to enable stopping operation if needed by depressing the button 182.

The user control pendant assembly 142 may include a pair of buttons 184 and 186 located on the top 172 of the housing 170 associated with and in communication with the switches, which are, in turn, in communication with the pendant controller. The buttons 184 and 186 allow the user to interface with the navigation screen/commands such as the system 10 of FIG. 1 if need be, or to utilize an accessory device 190 to be described in the event the accessory device 190 is coupled to the distal end 178 and is an active device such as an electrosurgical device, an ultrasonic device, a suction device, an irrigation device, etc. The buttons 184 and 186 may also be provided to control the feed rate of the surgical tool 22 as previously described. The user control pendant assembly 142 also includes an electrical wire 188 connected to the pendant controller and the manipulator controller 54 of FIG. 1 for power and information transfer.

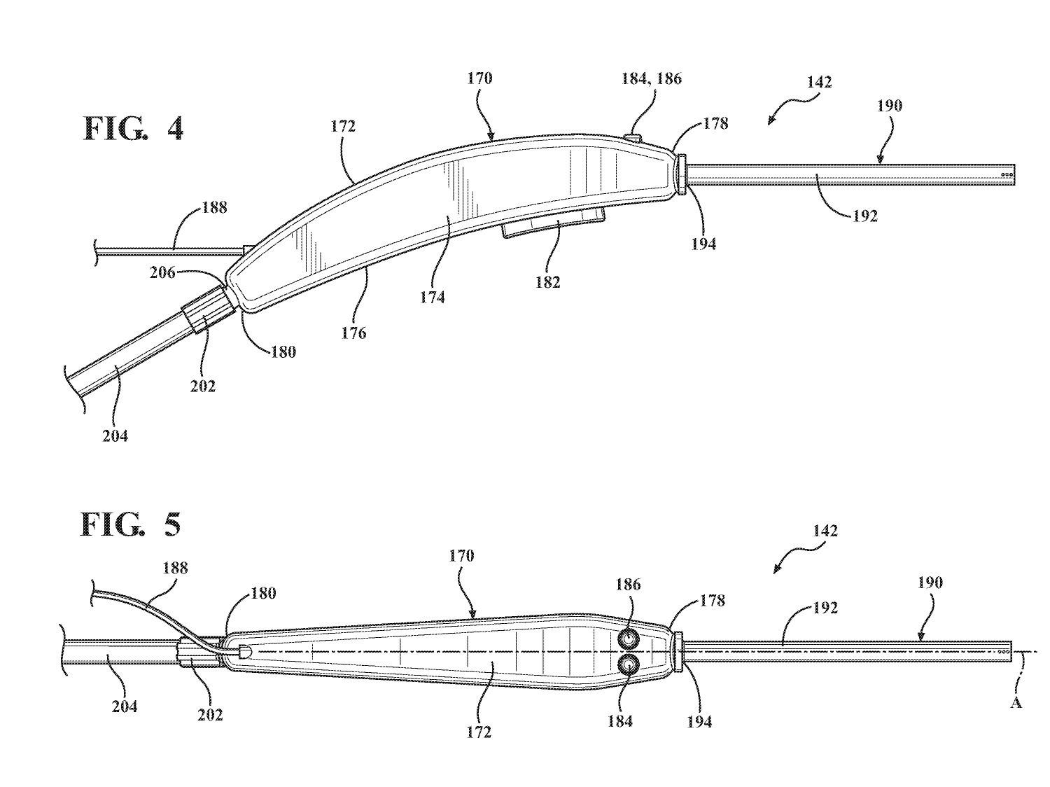

The user control pendant assembly 142 may include an accessory device, generally indicated at 190, coupled to the housing 170, such as at the distal end 178 of the housing 170. In one embodiment, the accessory device 190 is a suction tip. As illustrated in FIGS. 4-8, the accessory device 190 includes a conduit 192 extending axially along the axis A. In one embodiment, the conduit 192 is hollow and generally circular in cross-sectional shape. The conduit 192 may have aspiration apertures (not numbered) near its distal end to facilitate suction of bodily fluids, tissue, etc. from the surgical site. The conduit 192 may be flexible, rigid, or combinations thereof.

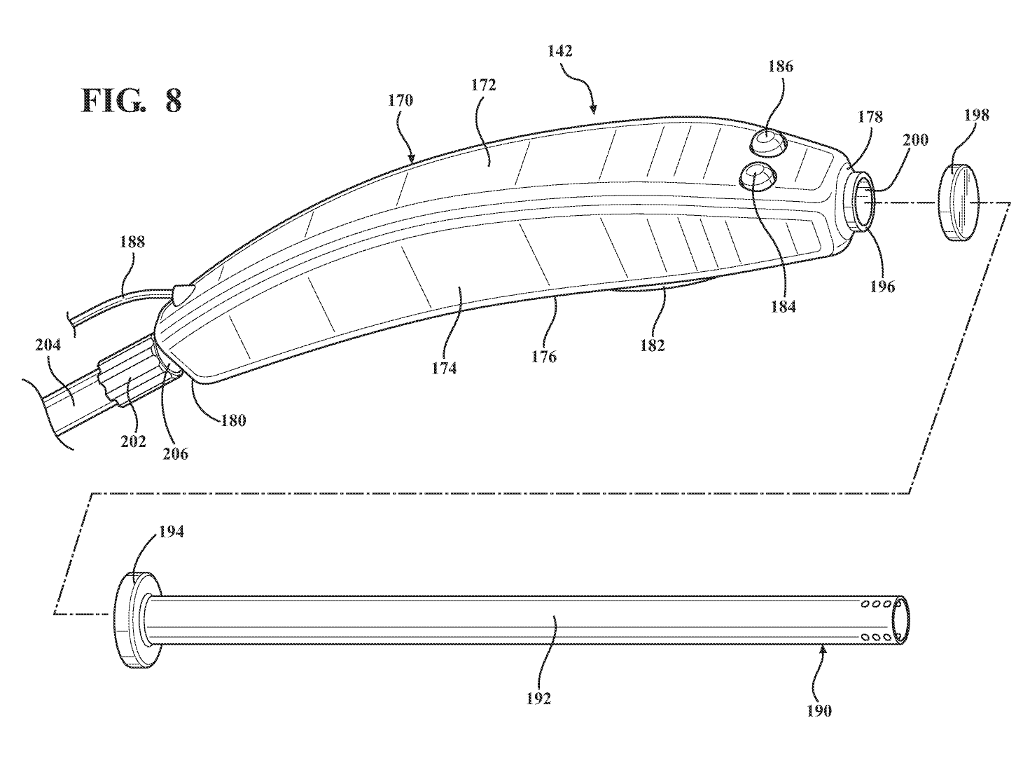

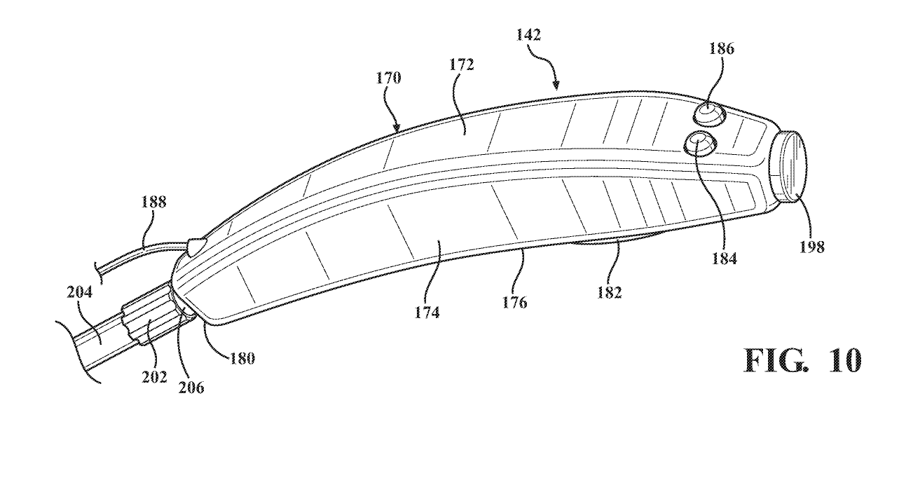

The accessory device 190 also has a coupling 194 at one end of the conduit 192 for coupling the conduit 192 to the distal end 178. In one embodiment, the distal end 178 of the housing 170 includes a projection 196 extending axially. In one embodiment, both the projection 196 and the coupling 194 are generally circular in cross-sectional shape, but may be any suitable shape. The coupling 194 is secured to projection 196 at the distal end 178 of the housing 170 by a suitable mechanism such as a friction fit, threaded connection, or the like. In one embodiment, the coupling 194 is made of a plastic material. As illustrated in FIGS. 8 and 10, the user control pendant assembly 142 may include a removable cap 198 coupled to the projection 196 when the accessory device 190 is not being used. In one embodiment, the cap 198 is generally circular in cross-sectional shape, but may be any suitable shape, and made of a plastic material. The cap 198 is secured to projection 196 at the distal end 178 of the housing 170 by a suitable mechanism such as a friction fit, threaded connection, or the like. In one embodiment, the coupling 194 is made of a plastic material. It should be appreciated that the cap 198 closes a passage 200 extending through the housing 170 at the distal end 178.

The user control pendant assembly 142 may include a coupling 202 at the proximal end 180 for connection to a suction conduit 204, which in turn, is connected to source of suction such as the source of suction 148 in FIG. 1 or the vacuum circuit 156 of FIG. 2. In one embodiment, the proximal end 180 of the housing 170 includes a projection 206 extending axially. In one embodiment, both the projection 206 and coupling 202 are generally cylindrical and circular in cross-sectional shape, but may be any suitable shape. The coupling 202 is secured to projection 206 at the proximal end 180 of the housing 170 by a suitable mechanism such as a threaded fit. In one embodiment, the suction conduit 204 is standard surgical suction tubing. It should be appreciated that the user control pendant assembly 142 is cannulated to be used as a suction device in the event a suction tip is clipped into the distal end 178. The user control pendant assembly 142 may include another removable cap (not shown) coupled to the projection 202 when the suction is not being used. It should be appreciated that the cap closes the passage 200 extending through the housing 170 at the proximal end 180.

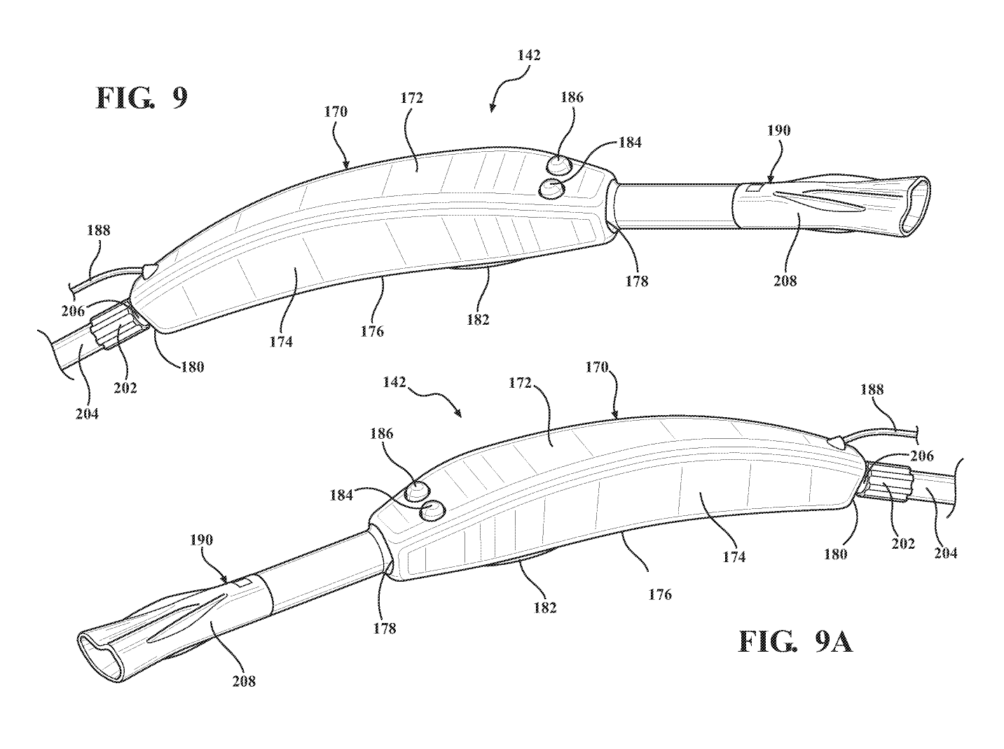

Referring to FIGS. 9 and 9A, another embodiment of the accessory device 190 is shown for the user control pendant assembly 142. In the embodiment illustrated, the accessory device 190 comprises an alternative tip 208. The tip 208 has a generally flattened shape at one end to get in the back of a joint (not shown) of a patient. In one embodiment, the tip 208 may be integrated with or removable from the housing 170 via a suitable mechanism such as a latch. In another embodiment, the accessory device 190 may be an electrosurgical cautery tool coupled to the housing 170. In other embodiments, the accessory device 190 may be an active device such as other electrosurgical devices, ultrasonic devices, suction devices, irrigation devices, etc. coupled to the housing 170. It should be appreciated that other types of accessories could be coupled to the housing 170 for the user control pendant assembly 142 such as a rake retractor, RF device, neuro monitoring probe, navigation pointer, ramen spectroscopy probe, light probe, irrigation coupling, etc. It should also be appreciated that the housing 170 may be re-usable and the tip 208 may be disposable or reuseable.

The user control pendant assembly 142 is cannulated and allows for different internal devices to extend in the passage 200 between the couplings 194 and 202 depending on the application. In one embodiment, the user control pendant assembly 142 may be cannulated and have a suction tube extending through the passage 200. In another embodiment, the user control pendant assembly 142 may be cannulated and have a bovie cord extending through the passage 200. In yet another embodiment, the user control pendant assembly 142 may be cannulated and have an RF cord extending through the passage 200. In still another embodiment, the user control pendant assembly 142 may be cannulated and have a neuro monitoring cord extending through the passage 200. In a further embodiment, the user control pendant assembly 142 may be cannulated and have a ramen spectroscopy probe extending through the passage 200. In still a further embodiment, the user control pendant assembly 142 may be cannulated and have a light cord extending through the passage 200. It should be appreciated that different internal devices may extend through the passage 200 depending on the accessory device 190 used.

Referring to FIG. 10, the user control pendant assembly 142 may be used without an accessory device 190. In this embodiment, the user control pendant assembly 142 has the suction tube 204, coupling 202, and accessory device 190 removed. The user control pendant assembly 142 is used as a simple handle/grip and includes the removable cap 198 to close the passage 200 at distal end 178 of the housing 170 and another removable cap to close the passage 200 at the proximal end 180 of the housing 170. It should be appreciated that the accessory device 190 does not have to be used at all, if the user only requires the user control pendant assembly 142 as a simple handle/grip, and can leave the removable cap 198 on the user control pendant assembly 142.

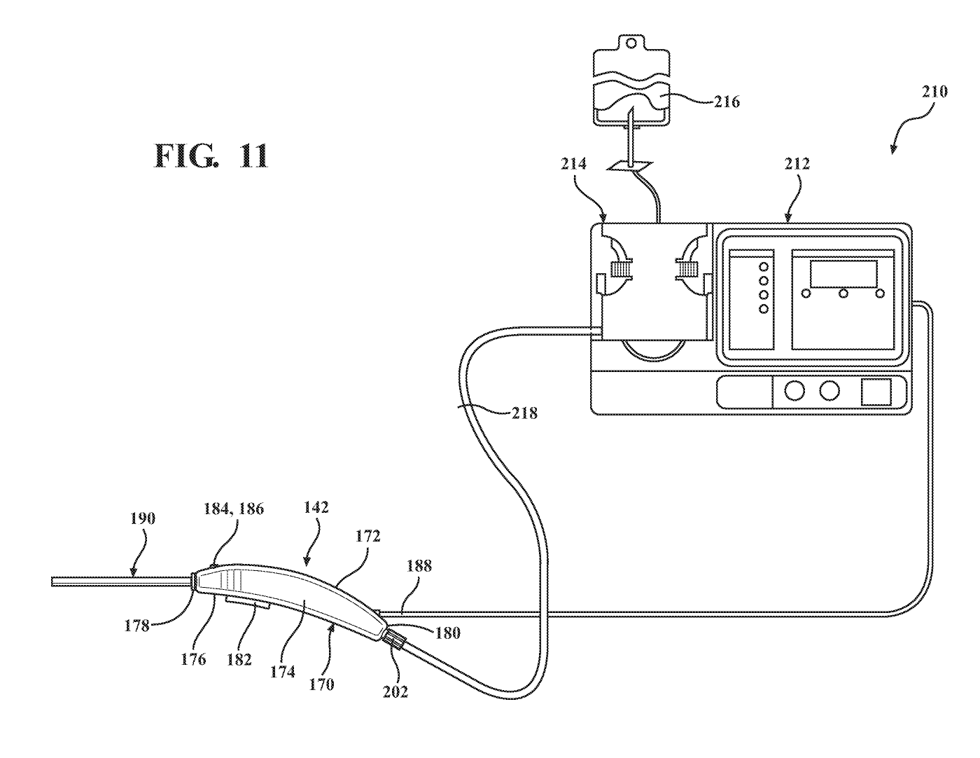

Referring to FIG. 11, in yet another embodiment, the user control pendant assembly 142 may be used with an irrigation source of an irrigation system. For example, in this embodiment, the user control pendant assembly 142 is connected to an irrigation system 210 to apply irrigation during a medical procedure while holding the user control pendant assembly 142 to control the manipulator 56 of FIG. 1. The irrigation system 210 includes a console, generally indicated at 212, located remote from the user control pendant assembly 142 and a tube set, generally indicated at 214, removably connectable with the console 212 and cooperable therewith for pumping irrigation liquid from a conventional irrigation liquid source, such as a conventional bag 216, through an irrigation tubing such as an outflow hose 218 to the user control pendant assembly 142. The user control pendant assembly 142 may include the coupling 202 at the proximal end 180 for connection to the outflow hose 218. An example of an irrigation system is discloses in U.S. Pat. No. 5,928,257 to Kablik et al., issued Jul. 27, 1999, entitled “Surgical Irrigation Pump and Tool System”, the entire disclosure of which is hereby incorporated by reference. It should be appreciated that the outflow hose 218 may be secured to the housing 170 of the user control pendant assembly 142 by any convenient releasable clip (not shown). It should also be appreciated that irrigation is fed through the passage 200 of the user control pendant assembly 142. It should further be appreciated that the buttons 184 and 186 could be employed to close or open flow through the user control pendant assembly 142.

In operation, the user control pendant assembly 142 allows a user to not only control the manipulator 56 (or other type of surgical device), but also to perform one or more ancillary functions, such as an electrosurgical function, ultrasonic function, suction function, irrigation function, and/or the like. In some cases, multiple accessory devices 190 may be coupled to the housing 170 simultaneously to perform multiple ancillary functions. In any case, when one or more accessory devices 190 are coupled to and/or integrated with the housing 170, the user is able to actuate one button, such as button 182 to start, stop, and/or continue operation of the manipulator 56 to perform the surgical procedure (e.g., cutting bone or other tissue from the patient), while simultaneously performing the one or more ancillary functions. This can be especially useful when the user is required to continually hold the user control pendant assembly 142 to enable continued operation of the manipulator 56, or other surgical device. Accordingly, for example, while the user holds the user control pendant assembly 142 and is actuating the button 182, the user can also be providing suction to the surgical site, irrigating the surgical site, cutting other tissue with an electrosurgical/ultrasonic tool from the surgical site, and the like. This provides additional efficiency in the operating room.

Several embodiments have been discussed in the foregoing description. However, the embodiments discussed herein are not intended to be exhaustive or limit the invention to any particular form. The terminology which has been used is intended to be in the nature of words of description rather than of limitation. Many modifications and variations are possible in light of the above teachings and the invention may be practiced otherwise than as specifically described.

What is claimed is:

1. A user control pendant assembly for remotely controlling a surgical device, the user control pendant assembly comprising:
a housing;
a plurality of user controls associated with said housing, one of said user controls being actuated by a user during operation of the surgical device; and
an accessory device coupled to said housing to allow a user to perform an ancillary function during operation of the surgical device, the ancillary function being ancillary to operation of the surgical device.

2. The user control pendant assembly as set forth in claim 1, wherein said one of said user controls is located at a bottom of said housing.

3. The user control pendant assembly as set forth in claim 1, wherein another one of said user controls is located at a top of said housing.

4. The user control pendant assembly as forth in claim 2, wherein said housing extends along an axis between a proximal end and a distal end.

5. The user control pendant assembly as set forth in claim 4, wherein said accessory device is coupled to said distal end.

6. The user control pendant assembly as set forth in claim 5, wherein said accessory device is a suction tip.

7. The user control pendant assembly as set forth in claim 6, wherein said suction tip comprises a conduit extending along an axis.

8. The user control pendant assembly as set forth in claim 7, wherein said suction tip includes a coupling for coupling said conduit to said distal end.

9. The user control pendant assembly as set forth in claim 6, including a suction tubing coupled to said proximal end.

10. The user control pendant assembly as set forth in claim 9, including a coupling for coupling said suction tubing to said proximal end.

11. The user control pendant assembly as set forth in claim 9, including a suction source coupled to said suction tubing.

12. The user control pendant assembly as set forth in claim 4, including an irrigation tubing coupled to said proximal end.

13. The user control pendant assembly as set forth in claim 12, including a coupling for coupling said irrigation tubing to said proximal end.

14. The user control pendant assembly as set forth in claim 12, including an irrigation source coupled to said irrigation tubing.

15. The user control pendant assembly as set forth in claim 1, wherein said accessory device is an active device.

16. The user control pendant assembly as set forth in claim 15, wherein said active device is one of an electrosurgical device, an ultrasonic device, a suction device, and an irrigation device.

17. The user control pendant assembly as set forth in claim 1, wherein said accessory device is an electrosurgical cautery tool coupled to said housing.

18. The user control pendant assembly as set forth in claim 1, wherein said accessory device is removably coupled to said housing.

19. The user control pendant assembly set forth in claim 1, including a removable cover cap coupled to said housing when said accessory device is not being used.

20. A robotic system comprising:
a manipulator;
a tool coupled to said manipulator and operable on a workpiece; and
a user control pendant assembly for remotely controlling said manipulator, said user control pendant assembly comprising a housing, a plurality of user controls associated with said housing, one of the user controls being actuated by a user during operation of said manipulator, and an accessory device coupled to said housing to allow a user to perform an ancillary function during operation of said manipulator, the ancillary function being ancillary to operation of said manipulator.
